(19) United States
(12) Patent Application Publication
(10) Pub. No.: US 2024/0238391 A1
(43) Pub. Date: Jul. 18, 2024

(54) TREATMENT OF THE DEMENTIAS USING BOTULINUM TOXIN

(71) Applicant: Penland Foundation, Beaumont, TX (US)

(72) Inventor: Roland M. Williams, Beaumont, TX (US)

(21) Appl. No.: 18/392,612

(22) Filed: Dec. 21, 2023

Related U.S. Application Data
(60) Provisional application No. 63/479,698, filed on Jan. 12, 2023.

(51) Int. Cl. A61K 38/48; A61K 9/00; A61P 25/28

(52) U.S. Cl. CPC A61K 38/4893; A61K 9/0019; A61K 9/0085; A61P 25/28

(57) ABSTRACT

Provided is a method of preventing a dementia in a patient in need thereof. The method may comprise administering a botulinum toxin to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating the dementia.

### CROSS-REFERENCE TO RELATED APPLICATION

This application claims the benefit of U.S. Provisional Application No. 63/479,698, filed Jan. 12, 2023, which is incorporated by reference herein in its entirety.

### BACKGROUND

### Technical Field

The present disclosure generally relates to a theory of the mechanism of the development of the dementias—the pathological activation of the cytokine system, which results in excess substance P which causes immune cells to chronically release cytokines. It also results in excess glutamate production which causes neuron death by neuron excitatory toxicity. The present disclosure also relates to the developmental mechanism of neural pruning and its potential involvement in the development of dementias. The present disclosure is also related to methods for treating, alleviating, and/or preventing this chronic over activation of the cytokine system, neuron excitatory toxicity, and neural pruning, which results in the dementias. Conditions involved include but are not limited to Alzheimer's, Parkinson's Disease, Vascular Dementia, Huntington's disease, Traumatic Brain Disease, and any neurological conditions that are caused by chronic elevation of substance P and glutamate levels.

### Background

Cytokine production is a normal part of infection control and damage repair. However, if overproduced, cytokines can damage or destroy neighboring neurons in the brain. Substance P activates the NK-1-3 receptors on immune cells, triggering the release of cytokines. Glutamate is the most widely used neurotransmitter in the brain, but its overproduction can damage or kill neurons by a condition called neuroexcitatory toxicity. Excess intracellular calcium can activate the developmental process of neural pruning. Botulinum toxin can mitigate the overproduction substance P and glutamate. Botulinum toxin can selectively stop only the chronic overproduction of substance P in the neurostructural cells in the sensory ganglia, thereby mitigating the premature damage and death of the brain neurons that cause dementia.

### SUMMARY

The present application proposes a novel theory on the cause of the dementias, an explanation of the symptoms of the dementias, and a practical treatment that can be applied safely in a clinical setting. The cause of dementia is not the plaque. The plaque is a result of the pathology. The techniques, locations, and dosing according to the present application allow botulinum toxin to be used safely and effectively in a clinical setting. Another observation that makes the treatment according to the present application effective is that any other dermatome can overproduce glutamate and that it can pass by passive diffusion in the spinal fluid to the brain. The location of injection and dosages according to the present application allow botulinum toxin to be injected safely with little or no side muscular side effects in all sensory dermatomes.

The present disclosure in some embodiments is related to a method of preventing a dementia in a patient in need thereof. The method may comprise administering a botulinum toxin to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating the dementia.

In some embodiments, the dementia may be associated with Alzheimer's disease, Parkinson's disease, Cardiovascular/Vascular dementia, Lewy Body disease, Huntington's disease, Traumatic Brain disease, Creutzfeldt-Jakob disease, HIV-associated dementia, Front temporal dementia, or a combination thereof.

In some embodiments, a therapeutically effective amount of the botulinum toxin may be 1-150 units. Preferably, a therapeutically effective amount of the botulinum toxin may be 1-60 units.

In some embodiments, the botulinum toxin may be selected from the group consisting of botulinum toxin type A, botulinum toxin type B, botulinum toxin type C, botulinum toxin type D, botulinum toxin type E, botulinum toxin type F and botulinum toxin type G, a fragment thereof, a hybrid thereof, a chimera thereof, and a combination thereof.

In some embodiments, a total dosage of the botulinum toxin to an adult who weighs about 150 lbs. may be less than or equal to about 50 units, and the total dosage of the botulinum toxin in an adult may be adjusted for weight.

In some embodiments, the trigeminal nerve may be selected from the group consisting of an ophthalmic nerve, maxillary nerve, mandibular nerve, supra orbital nerve, supra trochlear nerve, infraorbital nerve, lacrimal nerve, nasociliary nerve, superior alveolar nerve, buccal nerve, lingual nerve, inferior alveolar nerve, mental nerve, an auriculotemporal nerve, lesser occipital nerve, a greater occipital nerve and a combination thereof.

In some embodiments, the cervical nerve may be selected from the group consisting of a c-2 nerve, c-3 nerve, c-4 nerve, c-5 nerve, c-6 nerve, c-7 nerve, c-8 nerve and a combination thereof.

In some embodiments, the thoracic nerve may be selected from the group consisting of a t-2 nerve, t-3 nerve, t-5 nerve, t-6 nerve, t-7 nerve, t-8 nerve, t-9 nerve, t-10 nerve, t-11 nerve, t-12 nerve and a combination thereof.

In some embodiments, the lumbar nerve may be selected from the group consisting of a l-1 nerve, l-2 nerve, l-3 nerve, l-4 nerve, l-5 nerve and a combination thereof.

In some embodiments, the sacral nerve may be selected from the group consisting of a s-1 nerve, s-2 nerve, s-3 nerve, s-4 nerve, s-5 nerve and a combination thereof.

In some embodiments, each of the subcutaneous or intradermal injections may be bilateral.

In some embodiments, a maximum total dosage of the botulinum toxin may be 150 units.

### DETAILED DESCRIPTION OF THE PREFERRED EMBODIMENTS

Further in relation to this, before explaining at least the preferred embodiments of the invention in greater detail, it is to be understood that the invention is not limited in its application to the details of construction and to the arrangements of the components set forth in the following description. It would be understood by those of ordinary skill in the art that embodiments beyond those described herein are contemplated, and the embodiments can be practiced and carried out in a plurality of different ways. Also, it is to be understood that the terminology used herein is for the purpose of description and should not be regarded as a limiting factor.

Unless otherwise defined, the terms used herein refer to that which the ordinary artisan would understand such term to mean based on the contextual use of such term herein. To the extent that the meaning of a term used herein as understood by the ordinary artisan based on the contextual use of such term differs in any way from any particular dictionary definition of such term, it is intended that the meaning of the term as understood by the ordinary artisan will prevail.

As used herein, the term “about” means approximately or nearly and in the context of a numerical value or range set forth herein means 10% of the numerical value or range recited or claimed.

The term “treating” includes delaying, alleviating, mitigating or reducing the intensity, progression, or worsening of one or more attendant symptoms of a disorder or condition and/or alleviating, mitigating or impeding one or more causes of a disorder or condition. Treatment under the claimed invention may be a preventative treatment, prophylactic treatment, remission of treating or ameliorating treatment.

The term “therapeutically effective amount” or “therapeutically effective dose” refers to the amount of a composition, compound, therapy, or course of treatment that, when administered to an individual for treating a disorder or disease, is sufficient to effect such treatment for the disorder or disease. The “therapeutically effective amount” will vary depending on the composition, the compound, the therapy, the course of treatment, the disorder or disease and its severity and the age, weight, etc., of the individual to be treated.

The term “unit” refers to the amount of botulinum toxin needed to kill 50% of a group of 18-20 gm female Swiss-Webster mice given the injection intraperitoneally.

The term “vicinity of a nerve” refers to anywhere on the dermatome involved with the nerve.

As used herein, “consists essentially of” when used in conjunction with a composition means excluding other materials that contribute to mitigating cytokine overproduction, thereby treating dementias and related conditions that have resulted from the overproduction of cytokines. The objective of administering botulinum toxin is to treat the conditions by mitigating cytokine overproduction. With the language, other materials that contribute to the treatment that materially affect the basic and novel characteristics of the disclosure are not required and are potentially counterproductive because they may offset the treatment effect of botulinum toxin. In other words, the meaning of “consists essentially of” is tied to the objective and excludes materials (that contribute to the treatment) that are pharmaceutically active for the treatment and materially mitigate cytokine overproduction and thereby affecting the treatment of the conditions. Small traces that have little or no effect to the treatment as part of the embodiments of the presentation disclosure may exist in a composition that consists essentially of botulinum toxin under the definition because it would not materially affect its function and/or objective.

In accordance with the principles of the present invention, use of botulinum toxin to treat dementia is provided.

### Botulinum Toxin

Patients with symptoms of the dementia can be treated with botulinum toxin injections by the novel injection disclosed herein technique in the dermatomes that produce the excess glutamate. The technique allows for injection in all sensory dermatomes if needed that produce excess glutamate without reaching the maximum safe dosage of botulinum toxin.

Botulinum toxin can mitigate or control the chronic overproduction of substance P and glutamate in the spinal cranial and vagus nerve ganglia. Preventing the chronic release of substance P and glutamate from these neuro structural cells, can suppress or control the chronic inflammation in various parts of the brain which is believed to be a major factor in the dementia, and stop the production of intercellular and intracellular plaque which is the result of improper protein folding due to elevated intercellular pH. It also prevents activation of the embryonic neural pruning mechanism activated by excess intracellular calcium ions.

Botulinum toxins cleave and destroy a protein called synaptosomal nerve-associated protein 25 (“SNAP25”) and/or synaptobrevin (also called vesicle-associated membrane protein (“VAMP”)). Botulinum toxins A, C, and E cleave SNAP25 at different locations, and the destroyed protein cannot function until the cell makes new ones. Botulinum toxins B, D, F, and G cleave VAMP present at the cytoplasmic surface of the synaptic vesicle. The two important locations in the body where the proteins are found are at the terminals of the motor neurons (muscle) and in the cell membranes of astrocytes, glial cells, and satellite cells. These three cell types surround sensory neurons and form part of the blood-brain barrier. In motor nerves, to cause them to fire, vesicles of acetylcholine are moved from inside the motor neuron across the cell membrane at the synapse between the motor nerve and muscle fiber. Acetylcholine is released into the synapse and activated receptors in the muscle fiber, which contracts the muscle fiber. In sensory nerves, when a nerve is damaged from physical or mental injuries, the three aforementioned structural cells produce large amounts of substance P, Calcitonin Gene-Related Peptide (CGRP), and glutamate internally and the molecules are moved by vesicles to the cell membrane where the SNAP25 and/or VAMP moves it through the cell membrane into the cerebral spinal fluid (CSF) that surrounds the neurons. There the molecules bind to the receptor on the sensory nerves, causing the neuro excitatory effects. The molecules can also diffuse in the cerebral spinal fluid and influence other sensory nerves to become hyperactive, a process called central sensitization.

This mechanism of cleaving the SNAP25 and/or VAMP in muscles and sensory nerves causes the only known clinical effects of the botulinum toxins, which paralyzes muscles for 3-4 months until the cell grows a new protein. This effect has been used for decades for overactive muscles (cervical dystonia, blepharospasm, tic, Parkinson's, cerebral palsy, etc.), wrinkles in the face, excessive sweating, and overactive bladder.

In the sensory nerves, botulinum toxin has been used for migraines and depression. The effect of blocking the SNAP25 and/or VAMP in the glial, satellite, and astrocyte cells will remain for 5-9 months until these cells grow their new proteins. The important part of this is the botulinum toxin does not destroy cells and does not stop the normal production or effects of acetylcholine (muscles) or substance P, CGRP, or glutamate in sensory nerves. These facts give huge advantages over a monoclonal antibody which would eliminate all glutamate, CGRP, and substance P. Side effects would be disastrous. The receptor antagonists are also involved and may be problematic. They are not site-specific; they block glutamate, substance P, and CGRP everywhere. Too little glutamate, substance P, and CGRP is as problematic as too much. It is hard to regulate the oral or I.V. doses to obtain the correct reduction in areas that are too high in glutamate, substance P, and CGRP, without over-reduction in areas with normal levels.

The cleaving of the SNAP25 and/or VAMP allows small doses of botulinum toxin to be injected into specific muscles to calm the muscle's overreaction or paralyze the muscles temporarily if that is required. Or, if injected subcutaneously near unmyelinated sensory nerves, it can stop the overproduction of the sensory neuron excitatory compounds without affecting normal glutamate, substance P, and CGRP production and function. It is, however, noted that botulinum toxin is highly lethal. Botulinum toxin is the most toxic poison known. One molecule of botulinum toxin destroys one protein molecule of SNAP25 and/or VAMP. Its production, storage, and injection must be carried out with knowledge and care.

In particular, the mechanism of the sensory effect (stopping overproduction of glutamate, substance P, and CGRP) is as follows: almost all nerves in the human body are surrounded by a protective coating called myelin, which protects the nerve and makes neural conduction faster. It is difficult for botulinum toxin to penetrate the myelin. Just under the skin are some sensory pain nerves called C-fibers, which are unmyelinated. Research has shown that it is much easier for the botulinum toxins to penetrate these axons and diffuse up the axon to the cell body into the CSF and affect the SNAP25 and/or VAMP on the glial, satellite, and astrocyte cells. Subsequently, botulinum toxin destroys the SNAP25 and/or VAMP proteins and prevents the release of the excess substance P, CGRP, and glutamate that is involved in the neural injury response mechanism without affecting normal glutamate, substance P, and CGRP production, use, or receptors. An example of what goes wrong with the normal nerve mechanism is an infection of a nerve by the shingles virus. The infection damages the nerve, but does not kill it, or there would be no feeling (numbness). This causes a spike in the production of glutamate, substance P, and CGRP, which causes the well-known shingles pain and hypersensitivity. Over 2-3 months, the infection is controlled, the nerve heals, and the overproduction of the neuro excitatory chemical gets back to normal. However, sometimes, for unknown reasons, the overproduction does not get back to normal but remains high, with persisting severe chronic pain and hypersensitivity. Chronically overstimulated neurons can cause numerous problems depending on where the neurons are located. The neuron excitatory substances can travel up the spinal cord to the brain in the CSF and affect neurons there. This process is called Central Sensitization.

### Dementia

Dementia is not a disease but a general term for the impaired ability of various parts of the brain to function normally. The symptoms can vary widely from person to person depending on the parts of the brain involved, the levels, and the location in the brain of the damage caused. The severity ranges from the mildest stage, when the person's functioning is starting to be affected, to the most severe stage, when there's complete dependency on others for basic life activities. Although Dementia is more common as people gets older, it is not a part of normal aging.

Dementia take on many forms. Alzheimer's disease (the most common), Parkinson's disease, Cardiovascular/Vascular dementia, Lewy Body disease, Huntington's disease, Traumatic Brain disease, Creutzfeldt-Jakob disease, HIV-associated dementia, and Front temporal dementia.

### Alzheimer's Disease:

Alzheimer's disease is a type of degenerative brain disease that worsens over time. It is thought to begin 20 years or more before symptoms arise with changes in the brain are unnoticeable to the affected person and only after years of brain changes do individuals experience noticeable symptoms such as memory loss and language problems. Symptoms occur because the neurons in parts of the brain involved in thinking, learning and memory have been damaged or destroyed. As the disease progresses, neurons in other parts of the brain are damaged or destroyed also.
Prevalence, Incidence and Mortality (March 2021—Alzheimer's Association 2021 Alzheimer's Disease Facts and Figures)
   An estimated 6.2 million Americans ages 65 and older are living with Alzheimer's dementia in 2021.
      Ages 65-74 years: 1.72 million (27.6%)
      Ages 75-84 years: 2.25 million (36.1%)
      Ages 85 and older: 2.27 million (36.4%)
   More than 1 in 9 people (11.3%) age 65 and older has Alzheimer's dementia.
   Two-thirds of Americans over age 65 with Alzheimer's dementia (3.8 million) are women.
   Deaths due to Alzheimer's between 2000 and 2019 has more than doubled.
Causes
   The hallmark pathologies of Alzheimer's disease are the accumulation of the protein fragment beta-amyloid, called beta-amyloid plaques, outside neurons in the brain. Twisted strands or abnormal form of the protein tau, called tau tangles, inside neurons. These changes are accompanied by the death of the neurons and damage to brain tissues.
      Plaques and smaller accumulations of beta-amyloid called oligomers may contribute to the damage and death of neurons (neurodegeneration) by interfering with neuron-to-neuron communication at synapses.
      Tau tangles block the transport of nutrients and other essential molecules inside neurons.
      Although the complete sequence of events is unclear, beta-amyloid may begin accumulating before abnormal tau, and increasing beta-amyloid accumulation is associated with subsequent increases in tau.
      The presence of toxic beta-amyloid and tau proteins is believed to activate immune system cells in the brain called microglia. Microglia try to clear the toxic proteins as well as widespread debris from dead and dying cells. Chronic inflammation may set in when the microglia can't keep up with all the needs to be cleared. Atrophy or shrinkage of the brain occurs because of cell loss. Normal brain function is further compromised by the loss of the brain's ability to metabolize glucose.
Risk Factors
   Age
   Genetics
   Family History
Signs and Symptoms
   Memory loss that disrupts daily life
   Challenges in planning or solving problems
   Difficulty completing familiar tasks
   Confusion with time and place
   Trouble understanding visual images and spatial relationship
   New problems with words in speaking or writing
   Misplacing things and losing ability to retrace steps
   Decreased or poor judgment
   Withdrawal from work or social activities
   Changes in mood and personality
Current treatment
   Pharmacologic treatment
      None of the drugs available slow or stop the damage and destruction of neurons.
         Rivastigmine—temporarily improves cognitive symptoms by increasing neurotransmitters in the brain
         Galantamine—temporarily improves cognitive symptoms by increasing neurotransmitters in the brain
         Donepezil—temporarily improves cognitive symptoms by increasing neurotransmitters in the brain
         Memantine—blocks certain receptors in the brain from excess stimulation that can damage nerve cells. Glutamate receptor antagonist.
         Memantine combined with donepezil—blocks certain receptors in the brain from excess stimulation that can damage nerve cells and temporarily improves cognitive symptoms by increasing neurotransmitters in the brain
         Aducanumab—under FDA review for potential approval; only one that may potentially slow the progression.
         Antipsychotic drugs maybe prescribed to treat hallucinations, aggression, and agitation. However, research showed that these drugs are associated with increased risks of stroke and death in individuals with dementia.
   Non-pharmacologic treatment
      Computerized memory training
      Listening to favorite music to stir recall
      Using special lighting to lessen sleep disorder
   Active management to improve quality of life
      Appropriate use of available treatment options
      Effective management of coexisting conditions
      Providing well-trained family caregivers
      Coordination of care with physicians and other health care providers
      Participation of activities that are meaningful to the individual
      Having opportunities to connect with others living with dementia
      Becoming educated about the disease
      Planning for the future
Prognosis
   The rate of progression varies. Alzheimer's life expectancy is an average of 3-11 years after diagnosis. Prognosis usually depends on the person's age and how much the condition has progressed before diagnosis.
   Alzheimer's disease is the sixth-most common cause of death in the United States.
Co-morbidities
   Hypertension
   Osteoarthritis
   Depression
   Diabetes mellitus
   Cerebrovascular disease

### Parkinson's Disease

Parkinson's disease is a motor system disorder that leads to shaking, stiffness and difficulty with walking, balance, and coordination. Its symptoms usually begin gradually and gets worse over time. As the disease progress, people may have difficulty walking and talking. They may also have mental and behavioral changes, sleep problems, depression, memory difficulties, and fatigue. Both men and women can have Parkinson's disease, but the disease affects 50 percent more men than women. The precise cause of Parkinson's disease is unknown, but some cases are hereditary while others are thought to occur from a combination of genetics and environmental factors that trigger the disease.
Prevalence, Incidence and Mortality (neurology.org November 2021)
   The age-adjusted mortality increased from 5.4 (95% confidence interval [CI] 5.3-5.5) per 100,000 population in 1999 to 8.8 (95% CI, 8.7-8.9) per 100,000 population in 2019, with an average annual percent change of 2.4% (95% CI, 1.8%-3.0%).
   From 1999 to 2019, Parkinson's disease mortality increased significantly across all age groups, both sexes, various racial/ethnic groups, and different urban-rural classifications. The US states and District of Columbia with reported death rates all experienced an increase in mortality. Significant differences by sex and race/ethnicity were noted. Age-adjusted mortality rates were twice as high in men as in women and were greater in white individuals than those from other racial/ethnic groups.
      The death rate in the U.S. has gradually and significantly increased—by 63%—over two decades across all age groups, sexes, and racial and ethnic groups, according to a study based on nationwide mortality data.
      In the same period, men died twice as much from the disease as women, and a higher death rate was also seen among white people relative to those from other racial and ethnic backgrounds.
Causes
   The causes of Parkinson's disease are still unknown, although there are some evidence of genetics and environmental factors, or a combination of both.
   In Parkinson's disease, brain cells become damaged or die in the part of the brain that produces dopamine—a chemical needed to produce smooth, purposeful movement. People with Parkinson's also lose the nerve endings that produce norepinephrine, the main chemical messenger of the sympathetic nervous system, which controls many functions of the body, such as heart rate and blood pressure. The loss of norepinephrine might help explain some of the non-movement features of Parkinson's, such as fatigue, irregular blood pressure, decreased movement of food through the digestive tract, and sudden drop in blood pressure when a person stands up from a sitting or lying-down position.
   Many brain cells of people with Parkinson's contain Lewy bodies, unusual clumps of the protein alpha-synuclein. Scientists are trying to better understand the normal and abnormal functions of alpha-synuclein and its relationship to genetic mutations that impact Parkinson's disease and Lewy body dementia.
Risk Factors
   Age and gender
      Parkinson's disease is most commonly found in adults over the age of 50 (although diagnoses can occur in much younger people).
      Men also have a higher risk of Parkinson's disease than women.
      The actual links between any of these factors and Parkinson's disease are not completely understood.
   Genetics
      Scientists estimate that less than 10% of cases of Parkinson's disease are primarily due to genetic causes. The most common genetic effect that triggers Parkinson's disease is mutation in a gene called LRRK2. Mutations in alpha-synuclein have also been found to trigger Parkinson's, but these are quite rare. In most cases, no primary genetic cause can be found.
   Environmental
      Certain environmental factors, such as significant exposure to pesticides or certain heavy metals and repeated head injuries, can increase risk of Parkinson's. Most people do not have a clear environmental cause for their Parkinson's diagnosis, and because many years can pass between exposure to an environmental factor and the appearance of Parkinson's disease symptoms, the connection is often difficult to establish. However, it seems likely that environmental factors do influence the development of Parkinson's, perhaps particularly in people who also have a genetic susceptibility.
Symptoms
   Tremor—shaking that has a characteristic rhythmic back and forth motion
   Rigidity—muscle stiffness or a resistance to movement, where muscles remain constantly tense and contracted
   Bradykinesia-slowing of spontaneous and automatic movement that can make it difficult to perform simple tasks or rapidly perform routine movements
   Postural instability-impaired balance and changes in posture that can increase the risk of falls.
   Other symptoms may include difficulty swallowing, chewing, or speaking; emotional changes; urinary problems or constipation; dementia or other cognitive problems; fatigue; and problems sleeping.
Current Treatment and success
   Medications:
      Drugs that increase the level of dopamine in the brain
      Drugs that affect other brain chemicals in the body
      Drugs that help control nonmotor symptoms
   Levodopa, also called L-dopa.
      Nerve cells use levodopa to make dopamine to replenish the brain's dwindling supply.
      Usually, people take levodopa along with another medication called carbidopa.
   Carbidopa prevents or reduces some of the side effects of levodopa therapy—such as nausea, vomiting, low blood pressure, and restlessness—and reduces the amount of levodopa needed to improve symptoms.
   Carbidopa-levodopa (Sinemet, Rytary, Duopa)
      This medication, known as a dopamine precursor, is the most potent and effective medication for Parkinson's. Levodopa is absorbed by nerve cells in your brain and turned into the neurotransmitter dopamine, which helps replace the dopamine lost to Parkinson's. It is usually taken as a liquid or tablet and taken alongside other medications like benserazide or carbidopa that reduce the side effects of levodopa and prevent it from being broken down in the bloodstream before it gets to the brain.
   Dopamine
      These drugs mimic dopamine's effects in the brain, helping relieve Parkinson's symptoms. Their effects are similar to levodopa but milder, and they can be taken less frequently than levodopa. Options include pramipexole (Mirapex), ropinirole (Requip), and rotigotine (Neupro).
   MAO-B inhibitors to slow down an enzyme that breaks down dopamine in the brain
      rasagiline (Azilect), safinamide (Xadago), and selegiline (Eldepryl)
   COMT inhibitors to help break down dopamine
   Amantadine, an old antiviral drug, to reduce involuntary movements
      Amantadine is known as a NMDA antagonist (glutamate), although the exact way it works in your body is not yet fully understood. It is prescribed to help treat dyskinesias and “off episodes” in patients already taking levodopa-based medication. Dyskinesia is a side effect of Parkinson's disease that causes involuntary movements. “Off episodes” occur when the medication you regularly take does not work as well as it usually does.
   Anticholinergic drugs to reduce tremors and muscle rigidity
   Deep Brain Stimulation
   DBS is a surgical procedure that surgically implants electrodes into part of the brain and connects them to a small electrical device implanted in the chest.
   The device and electrodes painlessly stimulate the brain in a way that helps stop many of the movement-related symptoms of Parkinson's, such as tremor, slowness of movement, and rigidity.
   Other Therapies
   Physical, occupational and speech therapies
   Healthy diet and exercise
Prognosis
   Parkinson's disease is classified by stages, ranging from 1 to 5. Stage 5 is the most advanced. Advanced stages may increase the risk of health complications that can reduce lifespan.
   Parkinson's is not a fatal disease, meaning one does not die from it. Early detection is the key to helping reduce complications that can shorten life expectancy.
Comorbidities
   cerebrovascular disease
   hypertension
   diabetes
   chronic pulmonary disease
   paralysis
   dementia

### Vascular Dementia

Vascular dementia is the second most common form of dementia, after Alzheimer's disease. This dementia causes a decline in brain function, or cognitive abilities, beyond what is expected from the normal aging process. Dementia causes problems with memory, thinking, behavior, language skills, and decision making. Vascular dementia is caused by conditions that damage the blood vessels in the brain, depriving the brain of oxygen. This oxygen shortage inhibits the brain's ability to work as well as it should. Symptoms of vascular dementia can begin gradually or can occur suddenly, and then progress over time, with possible short periods of improvement. Vascular dementia can occur alone or be a part of a different diagnosis such as Alzheimer's disease or other forms of dementia. When an individual is diagnosed with vascular dementia, their symptoms can be similar to the symptoms of Alzheimer's.
Prevalence, Incidence and Mortality
   The prevalence increases linearly with age and varies greatly from country to country, ranging from 1.2 to 4.2% of people over 65 years old, even after adjustment for age and sex.
   The incidence is more homogeneous than prevalence and is estimated at 6-12 cases per 1,000 persons over 70 years per year.
   The mean duration of the disease is around 5 years and survival is less than for the general population and for AD.
Theories of Causes
   Vascular dementia is caused by a series of small strokes.
      A stroke is a disturbance in or blockage of the blood supply to any part of the brain. A stroke is also called an infarct. Multi-infarct means that more than one area in the brain has been injured due to a lack of blood.
      If blood flow is stopped for longer than a few seconds, the brain cannot get oxygen. Brain cells can die, causing permanent damage.
      When strokes affect a small area, there may be no symptoms. These are called silent strokes. Over time, as more areas of the brain are damaged, the symptoms of dementia appear.
      Not all strokes are silent. Larger strokes that affect strength, sensation, or other brain and nervous system (neurologic) function can also lead to dementia.
Risk factors for vascular dementia include
   Diabetes
   Hardening of the arteries (atherosclerosis) heart disease
   High blood pressure (hypertension)
   Smoking
   Stroke
Symptoms
   Difficulty performing tasks that used to be easy, such as paying bills
   Trouble following instructions or learning new information and routines
   Forgetting current or past events
   Misplacing items
   Getting lost on familiar routes
   Problems with language, such as finding the right word or using the wrong word
   Changes in sleep patterns
   Difficulty reading and writing
   Loss of interest in things or people
   Changes in personality, behavior, and mood, such as depression, agitation, and anger
   Hallucinations or delusions (believing something is real that is not)
   Poor judgment and loss of ability to perceive danger
Current Treatment and success
   There is no treatment to turn back damage to the brain caused by small strokes
   An important goal is to control symptoms and correct the risk factors. To prevent future strokes:
      Avoid fatty foods. Follow a healthy, low-fat diet
      Less than 1 to 2 alcoholic drinks a day.
      Keep blood pressure lower than 130/80 mm/Hg.
      Control of LDH cholesterol less than 70 mg/dL.
      Smoking cessation.
      Blood thinners, such as aspirin, to help prevent blood clots from forming in the arteries.
      Medicines to control aggressive, agitated, or dangerous behaviors.
Prognosis
   Some improvement may occur for short periods, but the disorder will generally get worse over time.
Comorbidities
   cerebrovascular disease
   atherosclerosis
   heart failure
   atrial fibrillation
   septicemia
   injuries
   lung diseases including chronic obstructive pulmonary disease
   urinary diseases

### Lewy Body Dementia

Lewy body dementia (LBD) is a disease associated with abnormal deposits of a protein called alpha-synuclein in the brain. These deposits, called Lewy bodies, affect chemicals in the brain whose changes, in turn, can lead to problems with thinking, movement, behavior, and mood. Lewy body dementia is one of the most common causes of dementia. Diagnosing LBD can be challenging. Early LBD symptoms are often confused with similar symptoms found in other brain diseases or in psychiatric disorders. Lewy body dementia can occur alone or along with other brain disorders.
Prevalence, Incidence and Mortality
   The incidence was 3.5 per 100 000 person-years overall and was higher in men than in women. The incidence of DLB increased with age ranging from 10.3 in persons aged 60 to 69 years, peaking at 44.5 in persons aged 70 to 79 years, and remaining high at 30.1 in persons aged 80 to 99 years. The incidence rate in men was markedly higher than in women after age 60 years.
Theories of Causes
   The precise cause of LBD is unknown, but scientists are learning more about its biology and genetics. For example, it is known that an accumulation of Lewy bodies is associated with a loss of certain neurons in the brain that produce two important chemicals that act as messengers between brain cells (called neurotransmitters). One of these messengers, acetylcholine, is important for memory and learning. The other, dopamine, plays an important role in behavior, cognition, movement, motivation, sleep, and mood.
Symptoms
   It is a progressive disease, meaning symptoms start slowly and worsen over time. The disease lasts an average of five to eight years from the time of diagnosis to death but can range from two to 20 years for some people. How quickly symptoms develop and change varies greatly from person to person, depending on overall health, age, and severity of symptoms.
   In the early stages of LBD, symptoms can be mild, and people can function fairly normally. As the disease advances, people with LBD require more help due to a decline in thinking and movement abilities. In the later stages of the disease, they often depend entirely on others for assistance and care.
      Cognitive symptoms of Lewy body dementia
         LBD causes changes in thinking abilities. These changes may include:
         Visual hallucinations or seeing things that are not present. Visual hallucinations occur in up to 80 percent of people with LBD, often early on. Nonvisual hallucinations, such as hearing or smelling things that are not present, are less common than visual ones but may also occur.
         Unpredictable changes in concentration, attention, alertness, and wakefulness from day to day and sometimes throughout the day. Ideas may be disorganized, unclear, or illogical. These kinds of changes are common in LBD and may help distinguish it from Alzheimer's disease.
         Severe loss of thinking abilities that interfere with daily activities. Unlike in Alzheimer's dementia, memory problems may not be evident at first but often arise as LBD progresses. Other changes related to thinking may include poor judgment, confusion about time and place, and difficulty with language and numbers.
      Movement problems and Lewy body dementia
         Some people with LBD may not experience significant movement problems for several years. Others may have them early on. At first, movement symptoms, such as a change in handwriting, may be very mild and easily overlooked. Movement problems may include:
         Muscle rigidity or stiffness
         Shuffling walk, slow movement, or frozen stance
         Tremor or shaking, most commonly at rest
         Balance problems and repeated falls
         Stooped posture
         Loss of coordination
         Smaller handwriting than was usual for the person
         Reduced facial expression
         Difficulty swallowing
         A weak voice
      Lewy body dementia and sleep
         Sleep disorders are common in people with LBD but are often undiagnosed. A sleep specialist can help diagnose and treat sleep disorders. Sleep-related disorders seen in people with LBD may include:
         REM sleep behavior disorder
         Excessive daytime sleepiness (sleeping two or more hours during the day)
         Insomnia
         Restless leg syndrome
      Behavioral and mood symptoms of Lewy body dementia
         Changes in behavior and mood are possible in LBD and may worsen as the person's thinking abilities decline. These changes may include:
         Depression
         Apathy, or a lack of interest in normal daily activities or events and less social interaction
         Anxiety and related behaviors, such as asking the same questions over and over or being angry or fearful when a loved one is not present
         Agitation, or restlessness, and related behaviors, such as pacing, hand wringing, an inability to get settled, constant repeating of words or phrases, or irritability
         Delusions, or strongly held false beliefs or opinions not based on evidence. For example, a person may think his or her spouse is having an affair or that relatives who long dead are still living.
         Paranoia, or an extreme, irrational distrust of others, such as suspicion that people are taking or hiding things
      Other symptoms of Lewy body dementia
         People with LBD can also experience significant changes in the part of the nervous system that regulates automatic functions such as those of the heart, glands, and muscles. The person may have:
         Changes in body temperature
         Problems with blood pressure
         Dizziness
         Fainting
         Frequent falls
         Sensitivity to heat and cold
         Sexual dysfunction
         Urinary incontinence
         Constipation
         A poor sense of smell
Current Treatment and success
   There's no cure for Lewy body dementia but many of the symptoms can improve with targeted treatments.
   Medications:
      Cholinesterase inhibitors. These Alzheimer's disease medications, such as rivastigmine (Exelon), donepezil (Aricept) and galantamine (Razadyne), work by increasing the levels of chemical messengers in the brain (neurotransmitters) believed to be important for memory, thought and judgment. This can help improve alertness and cognition and might reduce hallucinations and other behavioral problems. Possible side effects include gastrointestinal upset, muscle cramps and frequent urination. It can also increase the risk of certain cardiac arrhythmias. In some people with moderate or severe dementia, an N-methyl-d-aspartate (NMDA) receptor antagonist called memantine (Namenda) might be added to the cholinesterase inhibitor.
      Parkinson's disease medications. These medications, such as carbidopa-levodopa (Sinemet, Rytary, Duopa) can help reduce parkinsonian signs and symptoms, such as rigid muscles and slow movement. However, these medications can also increase confusion, hallucinations and delusions.
      Certain medications can worsen memory. Try to avoid over-the-counter sleep aids that contain diphenhydramine (Advil PM, Aleve PM) and medications used to treat urinary urgency such as oxybutynin (Ditropan XL).
      Antipsychotic drugs can cause severe confusion, severe parkinsonism, sedation and sometimes death. Very rarely, certain second-generation antipsychotics, such as quetiapine (Seroquel) or clozapine (Clozaril, Versacloz) might be prescribed for a short time at a low dose but only if the benefits outweigh the risks.
Prognosis
   Life expectancy of people with one of the LBD is reduced; following diagnosis it ranges on average from five to eight years.
Comorbidities
   Mental and behavioral disorders
   Diseases of the nervous system
   Diseases of the eye and adnexa
   Diseases of the circulatory, respiratory, and genitourinary systems
   Diseases of the skin and subcutaneous tissue
   Diseases of the musculoskeletal system and connective tissue occurred more frequently in the DLB group after multivariate adjustment.
   Depression
   Migraine
   Ischenic stroke

### The Dementias

### Alzheimer's, Parkinson's Disease, Vascular Dementia, Lewy Body Dementia, Huntington's Disease, Traumatic Brain Disease, Creutzfeldt-Jakob Disease (Mad Cow Disease), Down's Syndrome

### There are Five Major Causes of the Dementias:

### 1. Severe Genetic Aberrations

Ex: Down's Syndrome, Huntington's Disease
These severe genetic defects cause malfunction and death of neurons. They are not treatable until genetic engineering allows for replacement of the defected genes.

### 2. Prion Disease

E.g., Creutzfeldt-Jakob disease (Mad Cow Disease)
Prions are misfolded proteins that have the ability to transmit their misfolded nonfunctional shape onto the normal variant of the same protein. They are transmissible and always fatal.

### 3. Direct Injuries to the Brain

List of conditions:
   i. Repetitive concussions, stroke, operation, anemia, toxin exposure, brain infections caused by viruses, bacteria, parasites
These brain injuries cause direct death to neurons and to make things worse, the huge surge in glutamate levels and inflammation from the injury initiates additional damage to neighboring neurons.

### 4. Comorbidities

Anxiety and/or depression
Sleep disturbance
Diabetes
Cardiovascular disease
Hypertension
Osteoporosis
Osteoarthritis
Fibromyalgia
Migraines
The associated comorbidities can release glutamate into the cerebrospinal fluid. The excess glutamate can travel up the spinal cord by diffusion to the brain that can cause direct overstimulation of the brain. By the process called central sensitization, the neurons in the brain can be irritated from the excess glutamate this causes their neurostructural cells to also produce excess glutamate.

### 5. Post-Traumatic Stress Disorder (PTSD)

Studies have shown that mental injury (PTSD) can vastly elevate CSF and brain levels of glutamate. If acute levels do not return to normal after a traumatic event, chronic overproduction of glutamate occurs and dementia can result.
Studies have shown that all these conditions whether physical injury or mental injury (PTSD), associated with a comorbidity, share a common thread. They all involve acute or chronically elevated levels of glutamate and substance P.
   Defective Proteins are Associated with the Dementias
Lewy Bodies misfolded alpha-synuclein protein
   i. It is a protein in neurons that assist in neurotransmitter release, vesicle turnover, and double stranded DNA break repair. The defective proteins form clumps in the cytoplasm of neurons. They are found in dementia, Parkinson's, multiple system atrophy, Alzheimer's disease.
   ii. In forming memories, especially traumatic memories, DNA strands are broken to expose the DNA sequence for faster translation. This protein is very important in repairing these DNA break involved in memory retention (short term memory). This is the probable cause of short term memory disfunction in dementia patients.
Beta amyloid peptide (plaque outside neurons)
   i. Found outside and around neurons
   ii. Precursor peptides of amyloid beta precursor protein (app)
   iii. It is a transmembrane protein that is critical to neuron growth, survival, and injury repair.
   iv. It is embedded in the cell membrane. These defective peptides are extruded outside the cells where they build up and form the characteristic plaque of the dementias.
Tau protein
   i. Internally, each neuron has a cytoskeleton made-up of microtubular structures. These structures are involved in active transport of waste, nutrients, and other compounds inside the neuron and up and down the axons.
   ii. They are also structural in nature, supporting the shape of the neurons and axons.
   iii. In dementia, it forms tangles and clumps because of its malformed, non-functional folding.
   iv. Breakdown of the system of intracellular active transport of waste, protein, neurotransmitter, etc., is devastating to the function and life of the neuron.

The severe genetic defects will not be treatable until Genetic Replacement Therapy is possible. Likewise, the prion diseases are not treatable with current knowledge and techniques.

### Theory of the Cause of Neuron Misfunction and Death in the Dementias

There are genetic weaknesses that make development of a Dementia more likely, but they are not the cause of Dementia.
   The genetic mutations found in the late onset dementia are usually involved in glutamate signaling, dysfunctions in the ribosomes (protein folding), mitochondria function, or apoptosis mechanism that weakens the ability to respond to the excess calcium ions (ca²⁺) and resulting pH alteration brought on by the neuroexcitation effect of excess glutamate stimulation.

### Neuron Death is Caused by Three Factors:

### 1. Glutamate-Induced Neuroexcitatory Toxicity.

Elevated levels of glutamate are found in the brain and spinal cord of patients with the dementias.
The comorbidities are all associated with elevated brain and spinal fluid glutamate levels. The elevated glutamate levels cause varying levels of excessive firing of the neurons. The site in the brain where this occurs accounts for the varying and overlapping symptoms of the different dementias.
   i. For example:
      1. Alzheimer's disease—Frontal Cortex
      2. Parkinson's disease—the cerebellum (motor) parts of the brain.
The elevated level of firing of the neurons cause a build-up of calcium ions inside the neurons.
Intracellular pH is maintained in a very narrow range. The pH is maintained by a tightly regulated energy dependent buffering system.
At younger age, the elevated brain and spinal fluid glutamate levels cause the aforementioned comorbidities. As a person ages, the efficiency and function of the person's system begin to fail. Genetic weakness in the systems that works to manage the excess glutamate signaling comes into play. The excess firing and resulting build-up of Ca²⁺ ions inside the neuron cannot be controlled and the intracellular pH rises. This leads to cellular changes at the ribosomes. Constant normal pH is very important at the ribosome. Changes in pH leads to defective protein folding producing dysfunctional proteins. This is the cause of the defective proteins found in the dementias. They are not the cause of dementia—just a symptom of the condition.
The misfolded and defective Beta amyloid protein because it is embedded in the cell membrane is excreted outside the cell causing the plaques. The defective Tau protein and Lewy body protein build up intracellularly.
All dementias are associated with mitochondria dysfunction and death. The mitochondria are thought to be ancient bacteria captured in cells that manufacture energy for their host. They have circular DNA like bacteria and a different intracellular pH. There is a large energy demand on the mitochondria in an to attempt to maintain the cellular buffering system. This system's function is to control intracellular pH. This overworks the mitochondria and leads to damage. When mitochondria start to malfunction and proteins are being malformed, a mechanism called apoptosis can be triggered. This mechanism results in cellular “suicide”.
Besides the extra workload to produce energy (ATP) to help cellular buffering, calcium ions are one of the signaling molecules to activate more energy production by the mitochondria. This creates even more stress for the already overworked mitochondria.

### 2. Chronic Inflammation and Cytokine Production

Neuron lysis occurs due to excessive neuron death.
The body has a mechanism that disposes of dead or dying cells. If too many cells die at once, then the cells rupture spilling their toxic content.
When the cells die naturally or die through apoptosis, they are labelled by a protein called Protein S to be disposed of properly. This is accomplished by large white blood cells called phagocytes. They engulf the marked cells and digest them internally. However, if too many cells die at one time, the disposal system is overwhelmed. Instead of being digested internally in the phagocytes, the cells lyse. Their contents spill into the neighboring cytoplasm. These contents are toxic and cause an inflammatory response in the neurostructural cells (astrocytes, glial and satellite cells). Neurons are damaged. They produce substance P as a result of this damage. This is the second factor involved in neuron damage and death.
The chronic release of substance P causes cytokines from neighboring immune cells to further damage and kill the already stressed neurons.

### 3. Pathogenic Activation of the Neuron Pruning System

Neural pruning is a normal embryonic and brain development process by which the nonfunctioning or inefficient connection between neurons or the neurons themselves are removed. The mechanism by which this occurs is not totally understood but it is triggered by sudden increases in calcium in inefficient or unused axons and neurons.
During development it is estimated that up to 90% of the neural axons are removed because they are non-functional and not needed. Non-functional or inefficient neurons are also removed.
This neural pruning mechanism is triggered by cellular input of large levels of calcium ions. The chronic abnormal triggering of this embryonic and developmental mechanisms destroys normal neurons and functional axon interconnectors.

### Conclusion

In the dementias, excess glutamate and/or NMDA receptor dysfunction results in excess neural stimulation. This causes a build-up of calcium ions (ca²⁺) that alters intracellular pH. This elevated pH damage the mitochondria resulting to failure of the mitochondria by their effect to produce enough energy to maintain the intracellular buffering system.
At the ribosomes, the protein folding is very sensitive to pH levels. When pH changes, the result is misfolded, dysfunctional proteins.
These problems result in activation of the apoptosis and the embryotic pruning mechanism that result in neuron dysfunction and death.
If too many neurons die at once, this overwhelms the cellular disposal system resulting in some neurons lysing and spilling their toxic intracellular contents into the surrounding cytoplasm. This causes damage to the neighboring neurons. The damage triggers the neurostructural cell to produce more glutamate and substance P.
This excess glutamate causes even more stimulation and more intracellular calcium ions in neighboring neurons.
The excess substance P causes the immune cells to chronically release cytokines. This further damages or kills neighboring neurons.
The excess calcium can also trigger the embryonic and developmental neural pruning system which also damages and destroys neuron.

### Novel Treatment for Dementias

The present disclosure proposes to use botulinum toxin to control the excess glutamate production by the neurostructural cells in the spinal, trigeminal and vagal sensory ganglia. Studies show that they are the source of the excess glutamate.
The inventors have developed a novel site and an injection technique that allow for the safe use of botulinum toxin in all sensory dermatomes if necessary.
   Subcutaneous injections to reach the unmyelinated C-fiber nerves
   ½ inches lateral to the spine—it is a shorter distance to diffuse to the dorsal root ganglia. It is the only place in the body where the motor and sensory nerves are separated.
   Vagus nerve injection uses anastomosis between the cervical and trigeminal nerves and the Vagus nerve to reach the Vagus sensory ganglia with Botulinum Toxin
More than 60 units of botulinum toxin should control the excess neural firing and prevent cascade of events that lead to premature damage and death of brain neurons by the diffusion of cGK from other dermatomes and or central synthetization.
In acute injury, the excess glutamate and substance P that are released from the damaged and dying neurons is produced intraneural and excreted out the axon terminals. These levels are extreme, about 10-50× the normal levels. These acute levels damage and destroy the normal tissues surrounding the injured tissue. In some cases, the collateral damage to the injured nerves can be worse than the injury itself. After healing, the extreme glutamate levels sometimes do not return to normal and are the cause of a chronic inflammation state and resulting chronic neuron death. Blood levels of neuroexcitatory molecules such as glutamate or substance P can be monitored to make sure that the levels drop to normal, and the dementia symptoms can be monitored to make sure the symptoms normalize as well. When the botulinum toxin wears off, blood tests show an increase in glutamate and/or the symptoms begin to re-develop, more botulinum toxin can be given by injection to combat this effect. If levels/symptoms fail to normalize, then perhaps a small dose of one of the glutamate antagonists can be administered to help lower glutamate blood levels without producing side effects. For patients, as discussed, it is possible to use the claimed method to delay, alleviate, mitigate or reduce the intensity, progression, or worsening of one or more attendant symptoms of a disorder or condition, and/or the claimed method alleviates, mitigates or impedes one or more causes of a disorder or condition.

In some embodiments, a botulinum toxin may be administered to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating the dementia.

In some embodiments, a botulinum toxin may be administered to the patient by subcutaneous or intradermal injection, 2-4 units to and/or around the vicinity of a trigeminal nerve, 2-4 units to and/or around the vicinity of a cervical nerve, lateral to the patient's spine, 2-4 units to and/or around the vicinity of a thoracic nerve, lateral to the spine, 2-4 units to and/or around the vicinity of a lumbar nerve, lateral to the spine, and/or 2-4 units to and/or around the vicinity of a sacral nerve, lateral to the spine, thereby treating the dementia.

While the administration site is about 1 inch lateral to the patient's spine in the above embodiment, the distance can be more than 0 inches, about 0.1-3 inches, about 0.5-2.5 inches or about 1.0-2.0 inches. Alternatively, the distance can be about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 inches.

In some embodiments, the dementia may be associated with Alzheimer's disease, Parkinson's disease, Cardiovascular/Vascular dementia, Lewy Body disease, Huntington's disease, Traumatic Brain disease, Creutzfeldt-Jakob disease, HIV-associated dementia, Front temporal dementia, or a combination thereof.

In some embodiments, a therapeutically effective amount of the botulinum toxin may be 1-60 units.

In some embodiments, the botulinum toxin may be selected from the group consisting of botulinum toxin type A, botulinum toxin type B, botulinum toxin type C, botulinum toxin type D, botulinum toxin type E, botulinum toxin type F and botulinum toxin type G, a fragment thereof, a hybrid thereof, a chimera thereof, and a combination thereof.

In some embodiments, a total dosage of the botulinum toxin to an adult who weighs about 150 lbs. may be less than or equal to about 50 units, and the total dosage of the botulinum toxin in an adult may be adjusted for weight.

In some embodiments, the trigeminal nerve may be selected from the group consisting of an ophthalmic nerve, maxillary nerve, mandibular nerve, supra orbital nerve, supra trochlear nerve, infraorbital nerve, lacrimal nerve, nasociliary nerve, superior alveolar nerve, buccal nerve, lingual nerve, inferior alveolar nerve, mental nerve, an auriculotemporal nerve, lesser occipital nerve, a greater occipital nerve and a combination thereof.

In some embodiments, the cervical nerve may be selected from the group consisting of a c-2 nerve, c-3 nerve, c-4 nerve, c-5 nerve, c-6 nerve, c-7 nerve, c-8 nerve and a combination thereof.

In some embodiments, the thoracic nerve may be selected from the group consisting of a t-2 nerve, t-3 nerve, t-5 nerve, t-6 nerve, t-7 nerve, t-8 nerve, t-9 nerve, t-10 nerve, t-11 nerve, t-12 nerve and a combination thereof.

In some embodiments, the lumbar nerve may be selected from the group consisting of a l-1 nerve, l-2 nerve, l-3 nerve, l-4 nerve, l-5 nerve and a combination thereof.

In some embodiments, the sacral nerve may be selected from the group consisting of a s-1 nerve, s-2 nerve, s-3 nerve, s-4 nerve, s-5 nerve and a combination thereof.

In some embodiments, each of the subcutaneous or intradermal injections may be bilateral.

In some embodiments, a maximum total dosage of the botulinum toxin may be 150 units.

### Conclusion

The dementias (Alzheimer's, Parkinson's, etc.) are caused by the same mechanisms; the difference is which part of the brain is affected by the excess glutamate. The amount above normal of Glutamate levels. The brains inability to handle the excess stimulation which is caused by age, and genetic weakness in the involved mechanisms.

### Treatment

Botulinum toxin to control chronic blood and brain elevation of glutamate, substance P and cytokine that cause nerve death by neuroexcitatory toxicity and cytokine overproduction.

Botulinum toxins for use according to embodiments of the present disclosure can be stored in lyophilized, vacuum dried form in containers under vacuum pressure or as stable liquids. Prior to lyophilization, the botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

Preferably, the botulinum neurotoxin is peripherally administered by administering it to or in the vicinity of the aforementioned nerve or to the aforementioned nerve branch or its ganglion nuclei. This method of administration permits the botulinum neurotoxin to be administered to and/or to affect select intracranial target tissues. Methods of administration include injection of a solution or composition containing the botulinum neurotoxin, as described above, and include implantation of a controlled release system that controllably releases the botulinum neurotoxin to the target trigeminal tissue. Such controlled release systems reduce the need for repeat injections. Diffusion of biological activity of botulinum toxin within a tissue appears to be a function of dose and can be graduated. Jankovic J., et al Therapy with Botulinum Toxin, Marcel Dekker, Inc., (1994), page 150. Thus, diffusion of botulinum toxin can be controlled to reduce potentially undesirable side effects that may affect the patient's cognitive abilities. For example, the botulinum neurotoxin may be administered so that the botulinum neurotoxin primarily affects neural systems believed to be involved in a selected neuropsychiatric disorder and does not have negatively adverse effects on other neural systems.

In addition, the botulinum neurotoxin may be administered to the patient in conjunction with a solution or composition that locally decreases the pH of the target tissue environment. For example, a solution containing hydrochloric acid may be used to locally and temporarily reduce the pH of the target tissue environment to facilitate translocation of the neurotoxin across cell membranes. The reduction in local pH may be desirable when the composition contains fragments of botulinum neurotoxins that may not have a functional targeting moiety (e.g., a portion of the toxin that binds to a neurotoxin receptor, and/or a translocation domain). By way of example, and not by way of limitation, a fragment of botulinum toxin that comprises the proteolytic domain of the toxin may be administered to the patient in conjunction with an agent that decreases the local pH of the target tissue. Without wishing to be bound by any particular theory, it is believed that the lower pH may facilitate the translocation of the proteolytic domain across the cell membrane so that the neurotoxin fragment can exert its effects within the cell. The pH of the target tissue is only temporarily lowered so that neuronal and/or glial injury is reduced.

The botulinum toxin used in treating dementia in accordance with embodiments of the present disclosure comprises botulinum toxin type A, botulinum toxin type B, botulinum toxin type C, botulinum toxin type D, botulinum toxin type E, botulinum toxin type F, botulinum toxin type G, a fragment thereof, a hybrid thereof, a chimera thereof, or a combination thereof. Because of different mechanisms and cleavage sites of botulinum toxins, the potency, dosage, or duration may vary depend on the type of botulinum toxins. The botulinum toxin can be used with other modulating drugs or chemicals. In further embodiments, the therapeutically effective amount of the botulinum toxin administered is between about 1 unit and about 150 units. The therapeutically effective amount can be about 1 to about 50 units, about 1 to about 30 units, about 50 to about 100 units, about 1 to about 60, about 6 to about 60, and about 50 to about 150.

In some embodiments, a composition administered to a patient consists of botulinum toxin(s). Alternatively, a pharmaceutically active composition contained in a composition administered to a patient consists of botulinum toxin(s). The composition may additionally include, but not be limited to, a pharmaceutically inactive excipient, stabilizer and/or carrier. The composition may further comprise one or more additional pharmaceutically inactive ingredients. If lyophilized, the botulinum toxin may be reconstituted with saline or water to make a solution or composition to be administered to the patient. Alternatively, a composition administered to a patient comprises botulinum toxin(s) and other pharmaceutically active ingredients.

The composition may additionally include a pharmaceutically inactive composition such as a pharmaceutically inactive excipient, stabilizer and/or carrier.

The invention is further described in the following examples. These examples are for illustrative purposes only, and are not to be construed as limiting the appended claims. Examples 1-3 are prophetic examples, and Example 4 is an actual example.

### Example 1

A 75 year old male patient suffers from Alzheimer's disease with minor depression. The patient weighs about 170 lbs. The patient primarily has an issue with memory loss, confusion and general understanding or judgement. He receives botulinum toxin in the area of trigeminal, cervical, thoracic, lumbar and sacral nerves (2 units in ophthalmic, 2 units in maxillary, 2 units in mandibular of trigeminal nerve bilaterally; 2 units in the c-2-c-3, 2 units in the c-5-c-6, 2 units in the c-7-c-8 of cervical nerve bilaterally; 2 units in the t-1-t-3, 2 units in the t-5-t-6, 2 units in the t-8-t-9, 2 units in the t-11-t-12 of thoracic nerve bilaterally; 2 units in the l-1-l-2, 2 units in the l-3-l-4, 2 units in the l-4-l-5 of lumbar nerve bilaterally; 2 units in the s-1-s-2, 2 units in the s-3-s-4, 2 units in the s-5 of sacral nerve bilaterally for a total of not more than 60 units). The patient and his family report significant improvement in the brain function without administering medications such as Galantamine. The patient also shows lower level of depression after the treatment. After the botulinum toxin administration, glutamate blood levels are measured to be lower than before. The patient optionally receives additional treatments such as non-pharmacologic treatment and active exercise.

### Example 2

An 82 year old female patient suffers from Grade 2 Parkinson's disease with minor depression. The patient weighs about 155 lbs. The patient primarily has an issue with tremor, rigidity, and postural instability. She receives botulinum toxin in the area of trigeminal, cervical, thoracic, lumbar and sacral nerves (2 units in ophthalmic, 2 units in maxillary, 2 units in mandibular of trigeminal nerve bilaterally; 2 units in the c-2-c-3, 2 units in the c-5-c-6, 2 units in the c-7-c-8 of cervical nerve bilaterally; 2 units in the t-1-t-3, 2 units in the t-5-t-6, 2 units in the t-8-t-9, 2 units in the t-11-t-12 of thoracic nerve bilaterally; 2 units in the l-1-l-2, 2 units in the l-3-l-4, 2 units in the l-4-l-5 of lumbar nerve bilaterally; 2 units in the s-1-s-2, 2 units in the s-3-s-4, 2 units in the s-5 of sacral nerve bilaterally for a total of not more than 60 units). The patient and his family report significant improvement in the brain function without administering medications such as Levodopa or Dopamine. After the botulinum toxin administration, glutamate blood levels are measured to be lower than before. The patient optionally receives additional treatments such as deep brain stimulation and physical, occupational and speech therapies.

### Example 3

A 77 year old female patient suffers from vascular dementia. The patient weighs about 145 lbs. The patient primarily has an issue with sleep patterns, reading/writing and ability to perceive danger. She receives botulinum toxin in the area of trigeminal, cervical, thoracic, lumbar and sacral nerves (1 units in ophthalmic, 1 units in maxillary, 1 units in mandibular of trigeminal nerve bilaterally; 1 units in the c-2-c-3, 1 units in the c-5-c-6, 1 units in the c-7-c-8 of cervical nerve bilaterally; 1 units in the t-1-t-3, 1 units in the t-5-t-6, 1 units in the t-8-t-9, 1 units in the t-11-t-12 of thoracic nerve bilaterally; 1 units in the l-1-l-2, 1 units in the l-3-l-4, 1 units in the l-4-l-5 of lumbar nerve bilaterally for a total of not more than 30 units). After the botulinum toxin administration, glutamate blood levels are measured to be slightly lower than before. Additional 15 units of botulinum toxin are administered to the patient, and glutamate blood levels significantly drop to a normal level. The patient and his family report significant improvement in the brain function. For at least three months, there is no sign of getting worse with vascular dementia. The patient optionally corrects risk factors by avoiding fatty foods and alcoholic drinks, and maintaining pressure lower than 130/80 mm/Hg.

### Example 4

A 62 year old male patient suffered from moderate to severe Parkinson's disease diagnosed by physician. His symptoms have progressed over the last 20 years. He had no other medical conditions. His tremors were so bad that he cannot eat with utensils or drink from a cup.

Botulinum Toxin (type A) of 12 units in the branches of the trigeminal nerve (face) and 12 units along the cervical nerves (neck) were injected to the patient, according to the techniques described herein. The injections were made in accordance with the treatment system described herein for the injection of botulinum toxin.

After 10 days his tremors disappeared except when he was really tired, then just slight and hardly noticeable. The botulinum toxin (type A) lasted for 4 months. All symptoms returned when botulinum toxin (type A) wore off.

Botulinum toxin (type A) was injected using the same method: in 10 days, all symptoms disappeared except for the occasional slight tremors when he was really tired.

Patient experienced no side effects from injections. His only complaint was that his muscles were sore for a week after botulinum toxin (type A) worked. He thought that the muscles were sore because he over-exercised; he could do a lot of things he could not do before.

Patient quit taking beta blockers and L-Dopa medication because of side effects. He did this on his own because he felt he did not need them.

The death and dysfunction of the dopamine producing nerves in the cerebellum is caused by chronic sensory inflammation caused by excess glutamate production from the sensory nerves. The botulinum toxin's ability to stop this overproduction is believed to allow his remaining dopamine neurons to function normally. When the botulinum toxin wore off, the dysfunction returned.

The chronic inflammation can eventually destroy the neurons by the nueroexcitatory toxicity mechanism. The botulinum toxin treatment is believed to stop the progression of Parkinson's disease.

The usage of terms “may,” “can,” or similar terms herein are only for the purpose of flexibility in the disclosure and include, but are not limited to, the meaning of “is,” “are,” or similar terms.

Unless defined otherwise, all technical and scientific terms used herein have same meaning as commonly understood by the person of ordinary skill in the art to which this disclosure belongs.

It should be understood that the above description of embodiments of the invention and specific examples, while indicating preferred embodiments of the present invention, are given by way of illustration and not limitation. Many changes and modifications within the scope of the present invention may be made without departing from the spirit thereof, and the present invention includes all such changes and modifications.

What is claimed is:

1. A method of preventing a dementia in a patient in need thereof, the method comprising:
administering a botulinum toxin to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating the dementia.

2. The method of claim 1, wherein the dementia is associated with Alzheimer's disease, Parkinson's disease, Cardiovascular/Vascular dementia, Lewy Body disease, Huntington's disease, Traumatic Brain disease, Creutzfeldt-Jakob disease, HIV-associated dementia, Front temporal dementia, or a combination thereof.

3. The method of claim 1, wherein a therapeutically effective amount of the botulinum toxin is 1-60 units.

4. The method of claim 1, wherein the botulinum toxin is selected from the group consisting of botulinum toxin type A, botulinum toxin type B, botulinum toxin type C, botulinum toxin type D, botulinum toxin type E, botulinum toxin type F and botulinum toxin type G, a fragment thereof, a hybrid thereof, a chimera thereof, and a combination thereof.

5. The method of claim 1, wherein a total dosage of the botulinum toxin to an adult who weighs about 150 lbs. is less than or equal to about 50 units, and the total dosage of the botulinum toxin in an adult is adjusted for weight.

6. The method of claim 1, wherein the trigeminal nerve is selected from the group consisting of an ophthalmic nerve, maxillary nerve, mandibular nerve, supra orbital nerve, supra trochlear nerve, infraorbital nerve, lacrimal nerve, nasociliary nerve, superior alveolar nerve, buccal nerve, lingual nerve, inferior alveolar nerve, mental nerve, an auriculotemporal nerve, lesser occipital nerve, a greater occipital nerve and a combination thereof.

7. The method of claim 1, wherein the cervical nerve is selected from the group consisting of a c-2 nerve, c-3 nerve, c-4 nerve, c-5 nerve, c-6 nerve, c-7 nerve, c-8 nerve and a combination thereof;

8. The method of claim 1, wherein the thoracic nerve is selected from the group consisting of a t-2 nerve, t-3 nerve, t-5 nerve, t-6 nerve, t-7 nerve, t-8 nerve, t-9 nerve, t-10 nerve, t-11 nerve, t-12 nerve and a combination thereof.

9. The method of claim 1, wherein the lumbar nerve is selected from the group consisting of a l-1 nerve, l-2 nerve, l-3 nerve, l-4 nerve, l-5 nerve and a combination thereof.

10. The method of claim 1, wherein the sacral nerve is selected from the group consisting of a s-1 nerve, s-2 nerve, s-3 nerve, s-4 nerve, s-5 nerve and a combination thereof.

11. The method of claim 1, wherein each of the subcutaneous or intradermal injections is bilateral.

12. A method of preventing a dementia in a patient in need thereof, the method comprising:
administering a botulinum toxin to the patient by subcutaneous or intradermal injection, 1-4 units to and/or around the vicinity of a trigeminal nerve, 1-4 units to and/or around the vicinity of a cervical nerve, lateral to the patient's spine, 1-4 units to and/or around the vicinity of a thoracic nerve, lateral to the spine, 1-4 units to and/or around the vicinity of a lumbar nerve, lateral to the spine, and/or 1-4 units to and/or around the vicinity of a sacral nerve, lateral to the spine, thereby treating the dementia, wherein a maximum total dosage of the botulinum toxin is 150 units.

13. The method of claim 12, wherein the dementia is associated with Alzheimer's disease, Parkinson's disease, Cardiovascular/Vascular dementia, Lewy Body disease, Huntington's disease, Traumatic Brain disease, Creutzfeldt-Jakob disease, HIV-associated dementia, Front temporal dementia, or a combination thereof.

14. The method of claim 12, wherein the botulinum toxin is selected from the group consisting of botulinum toxin type A, botulinum toxin type B, botulinum toxin type C, botulinum toxin type D, botulinum toxin type E, botulinum toxin type F and botulinum toxin type G, a fragment thereof, a hybrid thereof, a chimera thereof, and a combination thereof.

15. The method of claim 12, wherein the trigeminal nerve is selected from the group consisting of an ophthalmic nerve, maxillary nerve, mandibular nerve, supra orbital nerve, supra trochlear nerve, infraorbital nerve, lacrimal nerve, nasociliary nerve, superior alveolar nerve, buccal nerve, lingual nerve, inferior alveolar nerve, mental nerve, an auriculotemporal nerve, lesser occipital nerve, a greater occipital nerve and a combination thereof.

16. The method of claim 12, wherein the cervical nerve is selected from the group consisting of a c-2 nerve, c-3 nerve, c-4 nerve, c-5 nerve, c-6 nerve, c-7 nerve, c-8 nerve and a combination thereof.

17. The method of claim 12, wherein the thoracic nerve is selected from the group consisting of a t-2 nerve, t-3 nerve, t-5 nerve, t-6 nerve, t-7 nerve, t-8 nerve, t-9 nerve, t-10 nerve, t-11 nerve, t-12 nerve and a combination thereof;

18. The method of claim 12, wherein the lumbar nerve is selected from the group consisting of a l-1 nerve, l-2 nerve, l-3 nerve, l-4 nerve, l-5 nerve and a combination thereof.

19. The method of claim 12, wherein the sacral nerve is selected from the group consisting of a s-1 nerve, s-2 nerve, s-3 nerve, s-4 nerve, s-5 nerve and a combination thereof.

20. The method of claim 12, wherein each of the subcutaneous or intradermal injections is bilateral.
